(19) United States
(12) Patent Application Publication
(10) Pub. No.: US 2016/0324400 A1
(43) Pub. Date: Nov. 10, 2016

(54) ENDOSCOPE ASSEMBLY CAPABLE FOR CARRYING OPERATION DEVICES EXTERNALLY

(71) Applicant: Wen-Chieh Wu, Taoyuan Hsien (TW)

(72) Inventor: Wen-Chieh Wu, Taoyuan Hsien (TW)

(21) Appl. No.: 14/703,901

(22) Filed: May 5, 2015

(51) Int. Cl. A61B 1/00; G01N 21/954; A61B 17/32; G02B 23/24; A61B 1/04; A61B 1/06

(52) U.S. Cl. CPC A61B 1/00087; A61B 1/04; A61B 1/06; A61B 17/320016; A61B 1/0014; A61B 1/0011; G02B 23/2476; G01N 21/954; A61B 2017/00296

(57) ABSTRACT

An endoscope assembly capable for carrying operation devices externally comprises a holding portion holdable by an operator; a scope body including a soft tube and a hard tube; an end of the soft tube connected to the holding portion and another end of the soft tube being connected to the hard tube; the soft tube being bendable and deformable; at least one operation unit attached to an outer side of the scope body; the operation unit being movable with the scope body to enter into the object to be cured and then is separable from the scope body for performing operations; and a carrier attached to the outer side of the scope body for retaining the operation unit to the scope body so that the operation unit is movable synchronously with the scope body; when entering into the object, the operation unit is pulled to separate from the scope body.

### FIELD OF THE INVENTION

The present invention relates to endoscopes, and in particular to an endoscope capable of carrying objects externally and the operation thereof. The present invention is suitable for being used in medicine and industry.

### BACKGROUND OF THE INVENTION

Conventionally, an endoscope includes at least one machine channel, a camera, a light source, an air channel or a liquid channel, etc. The endoscope is formed by a soft tube and a hard tube. The machine channel serves for receiving various kinds of medical operation devices so that with the movement of the scope body, the devices can be guided into an object and then to a place to be cured and operated. A camera equipped in a front end of the scope body has the effect of taking pictures to the portion to be cured with the illumination of a light installed to the scope body. The machine channel, camera, light source are installed at an outside surface of the hard tube.

In the prior art, the machine channel is located within the scope body, and thus the actions and moving ranges of operation devices within the machine channel are confined and not freely so that the operations are also confined. Many complicated and wide range operations cannot be performed.

Therefore, the object of the present invention is to provide a novel endoscope structure which can improve the defects above mentioned.

### SUMMARY OF THE INVENTION

Accordingly, to improve above defects, the present invention provides an endoscope capable of carrying objects externally and the operation thereof. The present invention cause that the operation unit is confined by the scope body after it is placed into an object to be checked or operated. Originally, the operation unit is attached to the outer surface of the scope body by using a carrier. Then they are guided into a predetermined position of an interior of the object to be checked. Then the operation unit is separated from the scope body by pulling the operation unit from the carrier. Therefore, in operation, the operation unit has a wide moving coverage and the operation can be performed vividly and freely.

To achieve above objects, the present invention provides an endoscope assembly capable for carrying operation devices externally, comprising: a holding portion which is holdable by an operator; a scope body including a soft tube and a hard tube; an end of the soft tube being connected to the holding portion and another end of the soft tube being connected to the hard tube; the soft tube being bendable and deformable; at least one operation unit attached to an outer side of the scope body; the operation unit being movable with the scope body to enter into the object to be cured and then is separable from the scope body for performing operations; and a carrier attached to the outer side of the scope body for retaining the operation unit to the scope body so that the operation unit is movable synchronously with the scope body; when entering into the object, the operation unit is pulled to separate from the scope body.

### BRIEF DESCRIPTION OF THE DRAWINGS

FIG. 1 is a structural view of the present invention showing that the carrier tightly encloses a small part of the scope body.

FIG. 2 is another structural view of the present invention showing that the carrier tightly encloses a large part of the scope body.

FIG. 3 shows that an operation unit has been attached to a small part of the scope body by using the carrier.

FIG. 4 shows that an operation unit has been attached to a large part of the scope body by using the carrier.

FIG. 5 shows a C sleeve according to the present invention.

FIG. 6 shows that the operation unit of the present invention has entered into a predetermined position for curing.

FIG. 7 shows that the operation unit according to the present invention has separated from the scope body.

FIG. 8 is a schematic view showing that the operation unit has cut a tumor according to the present invention.

FIG. 9 is a front view of the endoscope of the present invention.

FIG. 10 shows a general operation steps of the present invention.

FIG. 11 shows a lumpectomy according to the present invention.

### DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

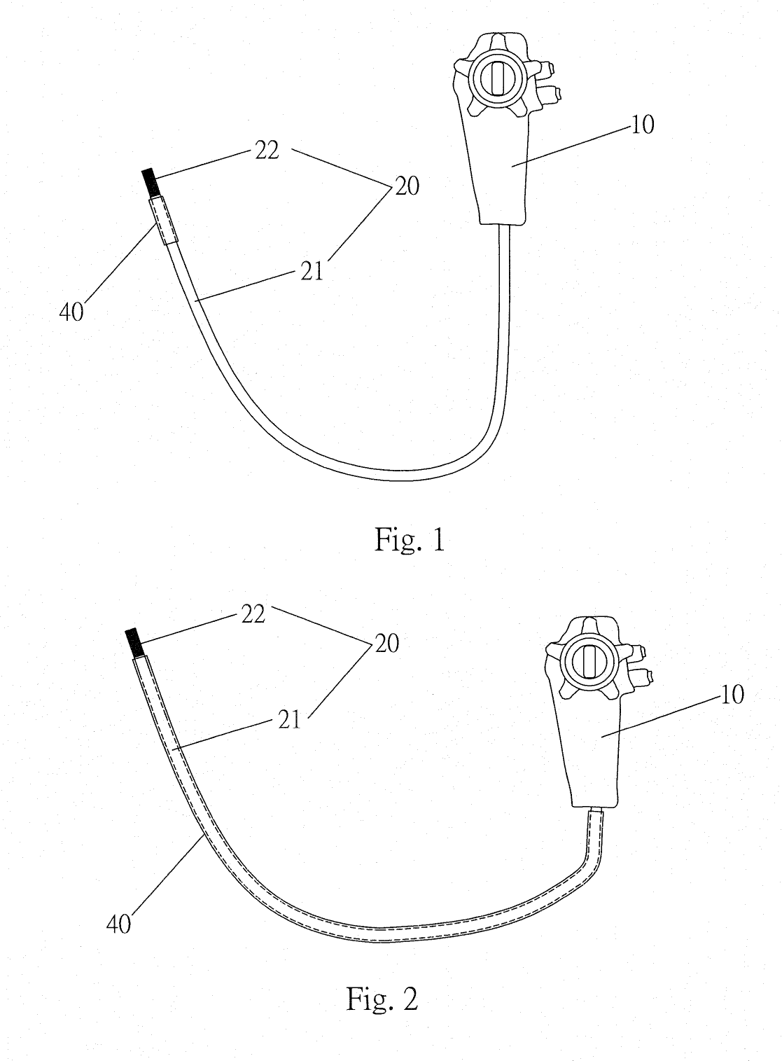

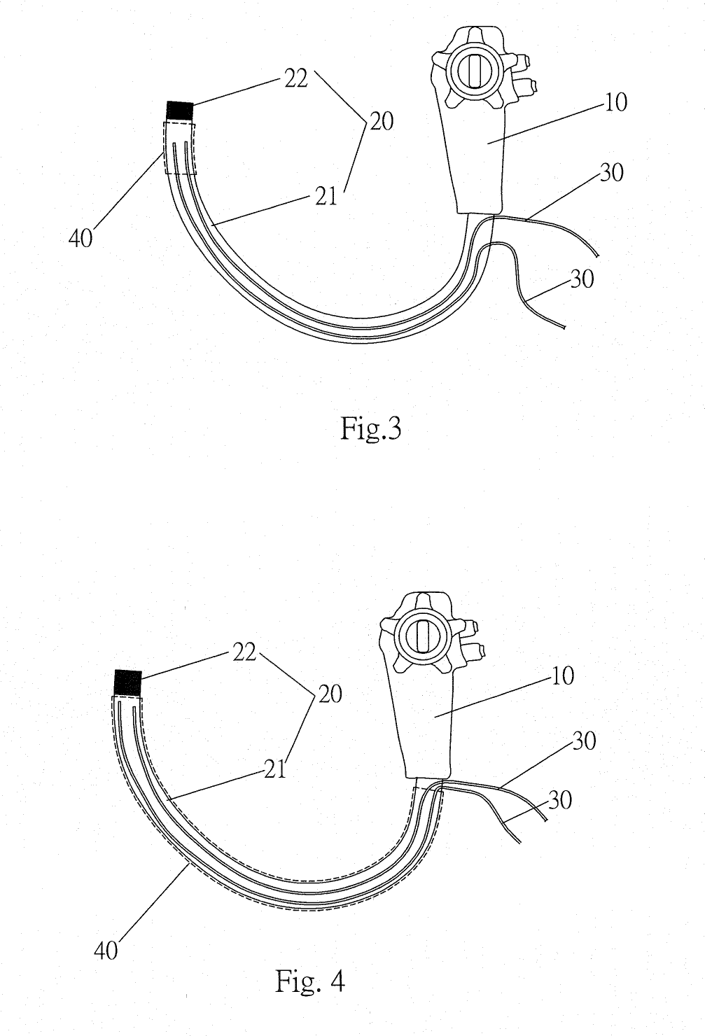

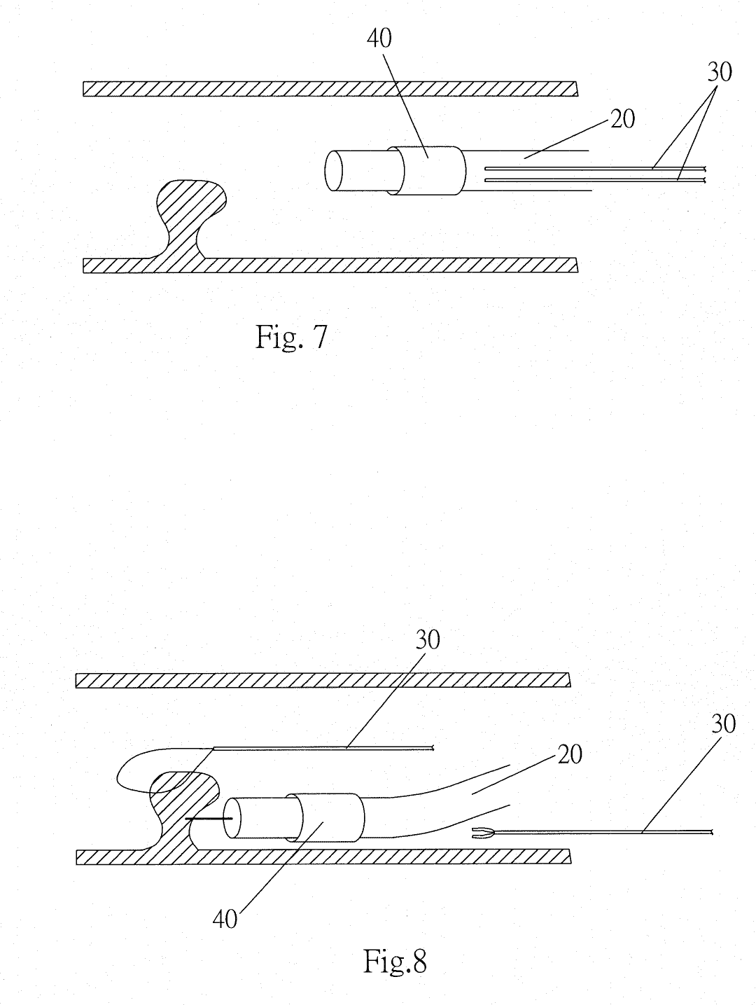

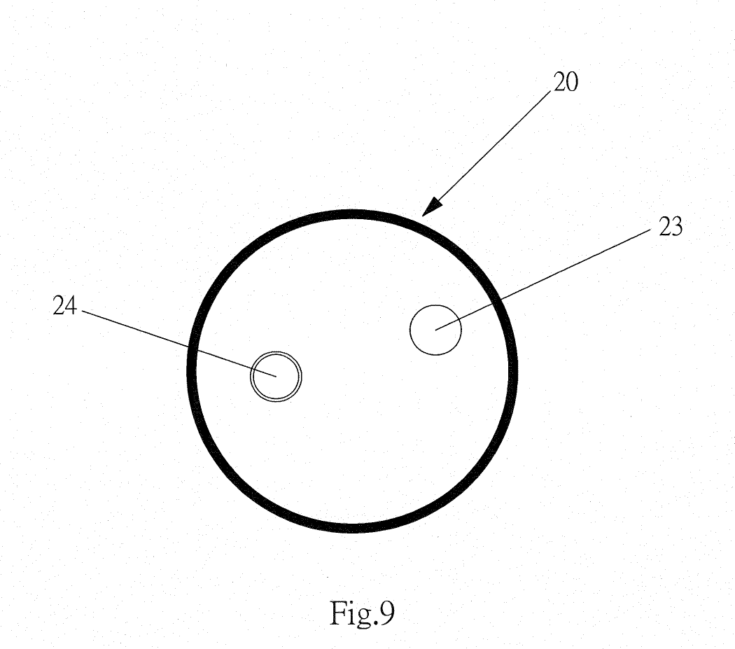

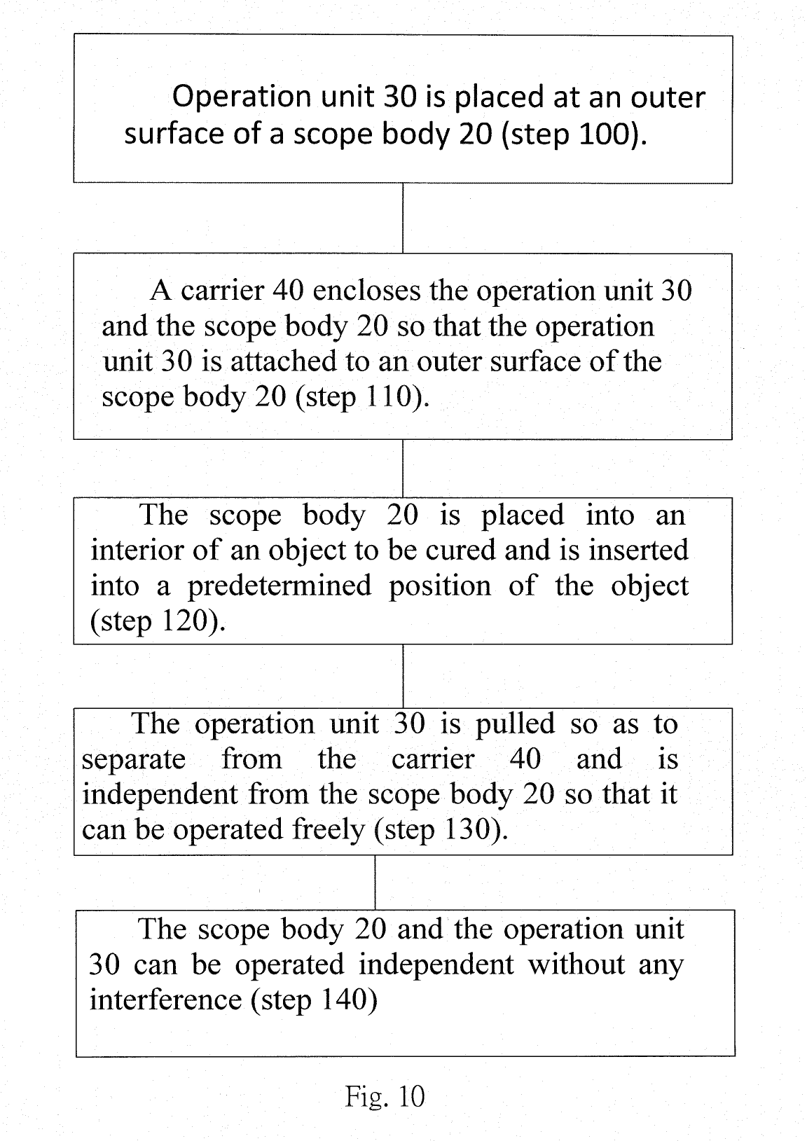

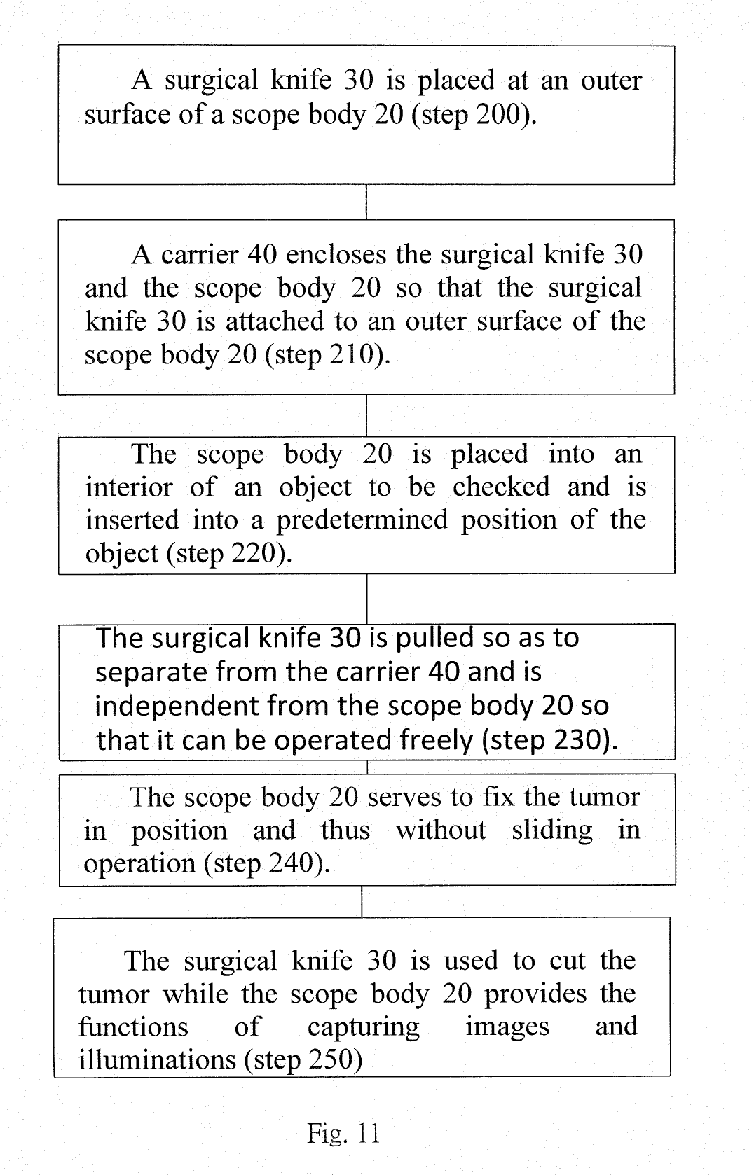

With reference to FIGS. 1 to 10, the structure of the present invention is illustrated. The present invention includes the following elements.

A holding portion 10 is to be held by an operator.

A scope body 20 includes a soft tube 21 and a hard tube 22. An end of the soft tube 21 is connected to the holding portion 10 and another end of the soft tube 21 is connected to the hard tube 22. The soft tube 21 is bendable and deformed easily. The scope body 20 is installed with a camera and a light source (not shown). A lens of the camera and an emission portion (not shown) of the light source are installed at an outer surface of the hard tube 22.

The first iron core 20 may be a gastroscope or a colonoscope.

At least one operation unit 30 is attached to an outer side of the scope body 20. The operation unit 30 is movable with the scope body 20 to enter into a body to be cured and then is separable from the scope body 20 for performing operations.

A carrier 40 is attached to the outer side of the scope body 20 for retaining the operation unit 30 to the scope body 20 so that the operation unit 30 is movable synchronously with the scope body 20. When the operation unit 30 is pulled properly, the operation unit 30 will separate from the scope body 20.

The carrier 40 encloses a small part or a large part of an outer side of the soft tube 21 of the scope body 20.

The carrier 40 may have different types. As illustrated in FIGS. 1 to 4, the carrier 40 is a sleeve which has a tubular structure for enclosing the outer surface of the scope body 20, while the operation unit 30 is placed between the sleeve 40 and the outer surface of the scope body 20 so as to be retained thereto and therefore move synchronously therewith.

In one embodiment of the present invention, the carrier 40 is a closed tubular structure with a circular cross section. The tubular structure may be combined by buckling, gluing, magnetic attraction, locking, etc.

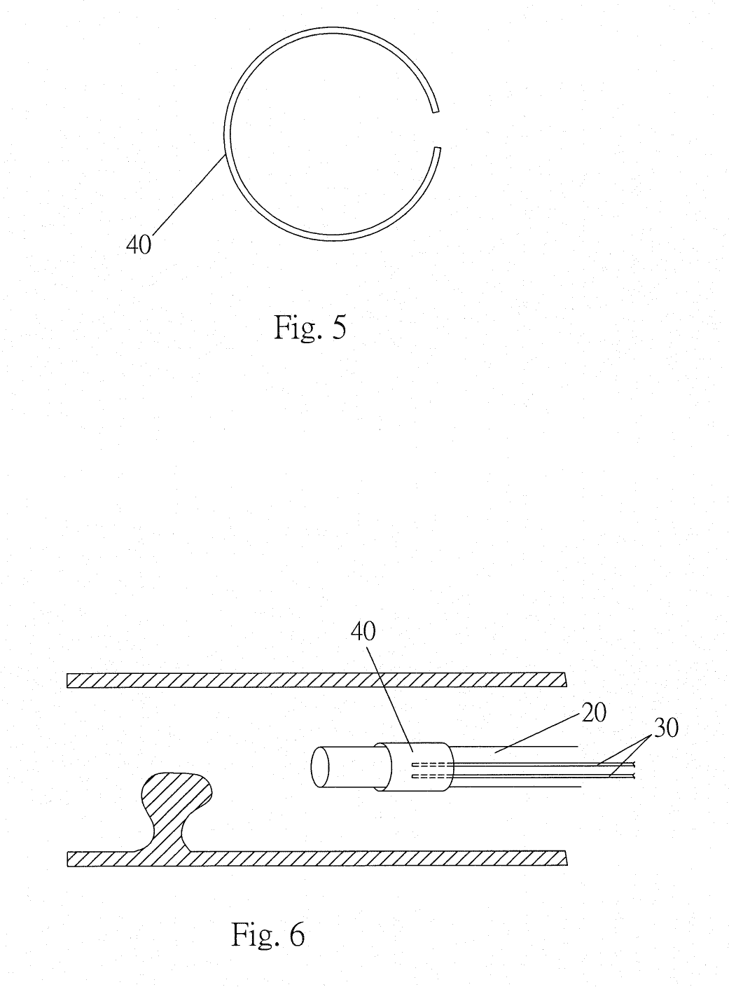

In another embodiment, as illustrated in FIG. 5, the carrier 40 is a C sleeve 40 which could enclose the outer surfaces of the scope body 20. The C sleeve 40 has a longitudinal notch and a C cross section. The operation unit 30 is clamped between the C sleeve 40 and the outer surface of the scope body 20. The operation unit 30 is movable synchronously with the C sleeve 40, while it is separable from the notch of the C sleeve 40.

The operation unit 30 is preferably a medical operation device and generally has a tubular structure.

The operation of the present invention will be described herein.

Firstly, the operation unit 30 is placed at an outer surface of the scope body 20 (step 100).

The carrier 40 encloses the operation unit 30 and the scope body 20 so that the operation unit 30 is attached to an outer surface of the scope body 20 (step 110).

The scope body 20 is placed into an interior of an object to be cured and is inserted into a predetermined position of the object (step 120). The operation unit 30 is pulled so as to separate from the carrier 40 and is independent from the scope body 20 so that it can be operated freely (step 130).

The scope body 20 and the operation unit 30 can be operated independent without any interference (step 140)

In above mentioned operation, other operation devices can be located within the scope body 20 and are guided by the scope body 20 to the predetermined position.

In the present invention, the operation unit 30 is guided by the scope body 20 and then the operation unit 30 is separated from the scope body 20 after they are guided to a predetermined position for operation. Therefore, because the two are separated, they are not interfered from each other and can be operated independently.

While in the prior art, the operation unit 30 is placed into the scope body 20 and then is guided to the position for operation, in operation, the operation unit 30 is stilled within the scope body 20, it is confined by the scope body 20 and thus is only movable within a finite and small coverage.

In the following, an embodiment about the operation of the present invention is described, wherein the present invention is used to the lumpectomy according to the present invention with reference to FIGS. 6 to 8 and 10.

Firstly, a surgical knife 30 is placed at an outer surface of the scope body 20 (step 200). The surgical knife is used as an operation unit in this embodiment.

The carrier 40 encloses the surgical knife 30 and the scope body 20 so that the surgical knife 30 is attached to an outer surface of the scope body 20 (step 210).

The scope body 20 is placed into an interior of an object to be checked and is inserted into a predetermined position of the object (step 220).

The surgical knife 30 is pulled so as to separate from the carrier 40 and is independent from the scope body 20 so that it can be operated freely (step 230). The scope body 20 and the surgical knife 30 can be operated independent without any interference.

With reference to FIG. 8, the scope body 20 serves to fix the tumor in position and thus without sliding in operation (step 240).

The surgical knife 30 is used to cut the tumor while the scope body 20 provides the functions of capturing images and illuminations (step 250)

In the present invention, the operation unit 30 may be various forms and thus serves for performing different surgeries, such as cutting of polyps, cutting of mucous membrane etc. Furthermore, according to the present invention, the number of the operation units to be carried 30 is not confined to one. Maybe, several operation units are carried by the scope body 20.

In above description, the endoscope is mainly used to medical operations, but this is not used to confine the scope of the present invention. The endoscope is also widely used in many industrial usages, especially, in electronic industry. By the endoscope, an interior of an object can be checked without detaching or explosion. Therefore, the present invention is used in all usages about endoscope including medical usages and industrial usages.

Advantages of the present invention are that the present invention cause that the operation unit is confined by the scope body after it is placed into an object to be checked or operated. Originally, the operation unit is attached to the outer surface of the scope body by using a carrier. Then they are guided into a predetermined position of an interior of the object to be checked. Then the operation unit is separated from the scope body by pulling the operation unit from the carrier. Therefore, in operation, the operation unit has a wide moving coverage and the operation can be performed vividly and freely.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An endoscope assembly capable for carrying operation devices externally, comprising:
a holding portion which is holdable by an operator;
a scope body including a soft tube and a hard tube; an end of the soft tube being connected to the holding portion and another end of the soft tube being connected to the hard tube; the soft tube being bendable and deformable;
at least one operation unit attached to an outer side of the scope body;
the operation unit being movable with the scope body to enter into the object to be cured and then is separable from the scope body for performing operations; and
a carrier attached to the outer side of the scope body for retaining the operation unit to the scope body so that the operation unit is movable synchronously with the scope body; when entering into the object, the operation unit is pulled to separate from the scope body.

2. The endoscope assembly capable for carrying operation devices externally as claimed in claim 1, wherein the carrier encloses a small part of an outer surface of the soft tube of the scope body.

3. The endoscope assembly capable for carrying operation devices externally as claimed in claim 1, wherein the carrier encloses a most part of the outer surface of the soft tube of the scope body.

4. The endoscope assembly capable for carrying operation devices externally as claimed in claim 1, wherein the carrier has a closed tubular structure with a circular cross section; the carrier is formed by circling a sheet and combining two side of the sheet by one of buckling, gluing, magnetic attraction, and locking.

5. The endoscope assembly capable for carrying operation devices externally as claimed in claim 1, wherein the carrier is a sleeve which has a tubular structure for enclosing the outer surface of the scope body, while the operation unit is placed between the sleeve and the outer surface of the scope body so as to be retained thereto and therefore move synchronously therewith.

6. The endoscope assembly capable for carrying operation devices externally as claimed in claim 1, wherein the carrier is a C sleeve capable of enclosing an outer surface of the scope body; and the C sleeve has a longitudinal notch and a C cross section; the operation unit is clamped between the C sleeve and an outer surface of the scope body so that the operation unit is movable synchronously with and separable from the C sleeve.

7. The endoscope assembly capable for carrying operation devices externally as claimed in claim 1, wherein the endoscope assembly is a medical endoscope.

8. The endoscope assembly capable for carrying operation devices externally as claimed in claim 1, wherein the endoscope assembly is an industrial endoscope.

9. An operation process for endoscope assembly capable for carrying operation devices externally including the following steps of placing an operation unit at an outer surface of a scope body of an endoscope assembly;
using an carrier to enclose the operation unit and the scope body so that the operation unit is attached to an outer surface of the scope body;
placing the scope body into an interior of an object to be cured and then inserted into a predetermined position of an object;
pulling the operation unit so that the operation unit separates from the carrier and is operable freely; and thus scope body and the operation unit are operated independently without any interference; and
wherein in above mentioned operation, operation devices can be located within the scope body and are guided by the scope body to the predetermined position.

10. The operation process as claimed in claim 9, wherein the scope body serves to fix a tumor in position without sliding in operation and the operation unit is a surgical knife which is used to cut the tumor, and the scope body also provides functions of capturing images and illuminations.

11. The operation process as claimed in claim 9, wherein the carrier encloses a small part of an outer surface of the soft tube of the scope body.

12. The operation process as claimed in claim 9, wherein the carrier encloses a most part of the outer surface of the soft tube of the scope body.

13. The operation process as claimed in claim 9, wherein the carrier has a closed tubular structure with a circular cross section; and the carrier is formed by circling a sheet and combining two side of the sheet by one of buckling, gluing, magnetic attraction, and locking.

14. The operation process as claimed in claim 9, wherein the carrier is a sleeve which has a tubular structure for enclosing the outer surface of the scope body, while the operation unit is placed between the sleeve and the outer surface of the scope body so as to be retained thereto and therefore move synchronously therewith.

15. The operation process as claimed in claim 9, wherein the carrier is a C sleeve capable of enclosing an outer surface of the scope body; and the C sleeve has a longitudinal notch and a C cross section; the operation unit is clamped between the C sleeve and an outer surface of the scope body so that the operation unit is movable synchronously with and separable from the C sleeve.

16. The operation process as claimed in claim 9, wherein the endoscope assembly is a medical endoscope.

17. The operation process as claimed in claim 9, wherein the endoscope assembly is an industrial endoscope.
